(19) United States
(12) Patent Application Publication
(10) Pub. No.: US 2010/0041998 A1
(43) Pub. Date: Feb. 18, 2010

(54) Method for Detecting and/or Monitoring a Wound Using Infrared Thermal Imaging

(76) Inventor: Olivier B. Postel, Redwood City, CA (US)

Correspondence Address: CENTRAL COAST PATENT AGENCY, INC, 3 HANGAR WAY SUITE D, WATSONVILLE, CA, 95076, US

(21) Appl. No.: 12/211,535

(22) Filed: Sep. 16, 2008

Related U.S. Application Data
(60) Provisional application No. 61/089,670, filed on Aug. 18, 2008.

(51) Int. Cl. A61B 6/00

(52) U.S. Cl. 600/475

(57) ABSTRACT

A system for diagnosing damaged tissue includes a thermal imaging device, a data processing device including a digital media generic to or associated there with connected to the thermal imaging device, an image display device connected to or integrated with the data processing device and a graphics user interface resident on the digital media of the data processing device and executable to display on the image display device, the interface enabling user configuration of various aspects of thermal imaging and data analysis functions.

### BACKGROUND OF THE INVENTION

1. Field of the Invention

The present invention is in the field of medical imaging systems and methods of use, and pertains more particularly to methods and apparatus for detecting and/or monitoring tissue wounds using thermal imaging.

2. Discussion of the State of the Art

In the art of medical imaging and at the time of this application, there are a variety of different technologies used in medical imaging. More recently, medical infrared imaging (MII) techniques such as digital infrared thermal imaging (DITI) have been used for early disease detection in specific medical diagnostic procedures such as detecting breast cancer. These techniques are broadly known in the medical field as Medical Thermography.

A thermography image is a color image representing a thermal radiation pattern recorded from detecting thermal radiation local to the site of a lesion or growth. In general the concept involves measuring changes in thermal radiation emanating from the targeted tissue. In cancer for example, the effected area will show a higher thermal signature because of higher metabolic activity due to increased blood and nutrient flow surrounding the growing node or tumor. Other medical conditions that result in some change of normal thermal radiation emitted in a localized fashion may be detected using thermal imaging cameras and display equipment.

One limitation of the current apparatus for thermal imaging is that the process is largely one-dimensional. The thermal signature represented by color image is also limited somewhat in resolution according to the quality of the infrared camera taking the measurements. For example, cameras that are not cooled during process provide much lower resolution than those cameras having cooling units or cells.

What is clearly needed is an improved apparatus and methods for digital medical thermal imaging. Such an improved system would enable more types of tissue conditions to be diagnosed and would improve prognostics and would enable more dimensional resolution relative to results for comparison analysis when monitoring wound recovery.

### SUMMARY OF THE INVENTION

The problem stated above is that monitoring the healing status of a wound is desirable for aiding recovery, but many of the conventional means for wound detection such as thermal imaging are not adapted for monitoring ongoing wound recovery. The inventors therefore considered functional elements of a thermal imaging system, looking for elements that exhibit modularity that could potentially be harnessed to provide thermal imaging but in a manner that would not limit to detection but that would enhance comprehensive diagnostic and prognostic capabilities.

Every thermal imaging system is adapted to detect thermal radiation emitted from an inanimate or animate object having a temperature above absolute 0 degrees and is adapted to produce one or more images of the object the image showing the thermal radiation pattern of the object. The image is a snapshot in time and does not characterize and evolution or change in the thermal radiation emitting from the object over time. A thermal imaging system in medical thermography or a thermograph employs an infrared camera for the purpose of detecting any anomalies in typical thermal patterns in a scan of a general area to discover a lesion or growth mostly associated with a chronic disease.

The present inventor realized in an inventive moment that if, at the point of imaging, multiple detectors could be employed from different angles significant dimensional improvement might result in thermal images rendered. The inventor subsequently realized also that the thermal progression of radiation released from a localized area of damaged tissue over time might be quantified to produce useful prognosis data covering different types of wound treatment therapies. The inventor therefore constructed a unique thermal imaging system for imaging damaged tissue such as in a wound that allowed tri-dimensional infrared modeling of a wound in one embodiment and ongoing prognosis of wound recovery determined from image analysis of multiple images rendered of the same wound over time. A significant improvement in diagnostic and prognostic capabilities results with no inconveniences or any residual side effects created.

Accordingly in one embodiment of the present invention a system for diagnosing damaged tissue is provided, comprising a thermal imaging device, a data processing device including a digital media generic to or associated there with connected to the thermal imaging device, an image display device connected to or integrated with the data processing device, and a graphics user interface resident on the digital media of the data processing device and executable to display on the image display device, the interface enabling user configuration of various aspects of thermal imaging and data analysis functions.

In one embodiment the imaging device is a digital camera with at least one infrared detector, the data processing device is a computer processor tower and the display device is a connected computer monitor. In one embodiment the damaged tissue being diagnosed is externally visible. In another embodiment, the damaged tissue being diagnosed is underneath the skin and not visible. In one embodiment the system is used to detect the damaged tissue before imaging.

In a preferred embodiment, the thermal imaging device is sensitive to thermal radiation from 0.07 microns in the near infrared range up to 9 microns in the far infrared range. In one embodiment the connection between the processing device and the thermal imaging device is a data cable. In this embodiment the cable is one of a universal serial bus (USB) cable, a fire wire cable, an Institute of Electrical and Electronic Engineers (IEEE) cable or a Super Video (S-Video) cable.

In another embodiment of the present invention the thermal imaging system further includes at least one additional imaging device, and a mounting bracket or mechanism for facilitating adjustable mounting of the imaging devices about the wound. In a variation of this embodiment the mechanism to which the cameras are mounted to is a goniometer track. In one embodiment employing multiple imaging devices, the imaging devices are digital cameras with at least one infrared detector, the data processing device is a computer processor tower and the display device is a connected computer monitor.

In one embodiment employing multiple imaging devices, the damaged tissue is externally visible. In another embodiment employing multiple imaging devices the damaged tissue is underneath the skin and not visible. In this embodiment the system is used to detect the damaged tissue before imaging.

In a preferred embodiment employing multiple imaging devices the thermal imaging devices are sensitive to thermal radiation from 0.07 microns in the near infrared range up to 9 microns in the far infrared range.

According to yet another embodiment of the invention a method for thermal imaging of damaged tissue is provided comprising the steps (a) powering on a thermal imaging system, the system including at least one thermal imaging device, (b) locating the damaged tissue to be imaged, (c) positioning the imaging device or devices over the tissue to be imaged, and (d) recording the thermal images.

In one aspect of the method the thermal imaging system also includes a computer processing tower, a connected monitor, and a graphics user interface. In one aspect the system is used to locate the damaged tissue, the tissue not visible to the operator of the system. In one aspect of the method an additional step is inserted between step (b) and step (c) for pre-treating the wound using a temperature controlled glove or boot. In another aspect of the method in step (c) the devices are positioned around the wound on a goniometer track and at step (d) multiple image recordings are made from the devices at different positions.

In one embodiment of the system the connection between the thermal imaging device and the data processing device is a wireless connection the imaging data transmitted to the data processing system from the thermal imaging device over the connection. In another aspect of the method using a temperature controlled glove or boot, the temperature controlled glove or boot is used to produce an artificial temperature state at the wound site the transition from which back to the actual thermal state of the wound is monitored with respect to time of completion of the transition and analyzed for comparative results.

In another aspect of the system, the damaged tissue is illuminated during thermal imaging to improve image contrast. In the embodiment using more than one imaging device the damaged tissue is also illuminated during thermal imaging to improve image contrast. In an aspect of the method, a step is added between steps (c) and (d) for illuminating the damaged tissue to improve image contrast.

In one aspect of the system a temperature controlled glove or boot is used to produce an artificial temperature state at the wound site the transition from which back to the actual thermal state of the wound is monitored with respect to time of completion of the transition and analyzed for comparative results. This aspect may also apply to the embodiment of the system using multiple imaging devices.

### BRIEF DESCRIPTION OF THE DRAWING FIGURES

FIG. 1 is an architectural view of a digital thermal imaging system according to an embodiment of the present invention.

FIG. 2 is an architectural view of a digital thermal imaging system according to another embodiment of the present invention.

FIG. 3 is a process flow diagram illustrating steps for thermal imaging of a wound according to one embodiment of the present invention.

FIG. 4 is a process flow chart illustrating steps for thermal imaging according to another embodiment of the present invention.

FIG. 5 is a process flow chart illustrating steps for thermal imaging according to another embodiment of the invention.

FIG. 6 is a process flow chart illustrating steps for thermal imaging according to another embodiment of the invention.

### DETAILED DESCRIPTION

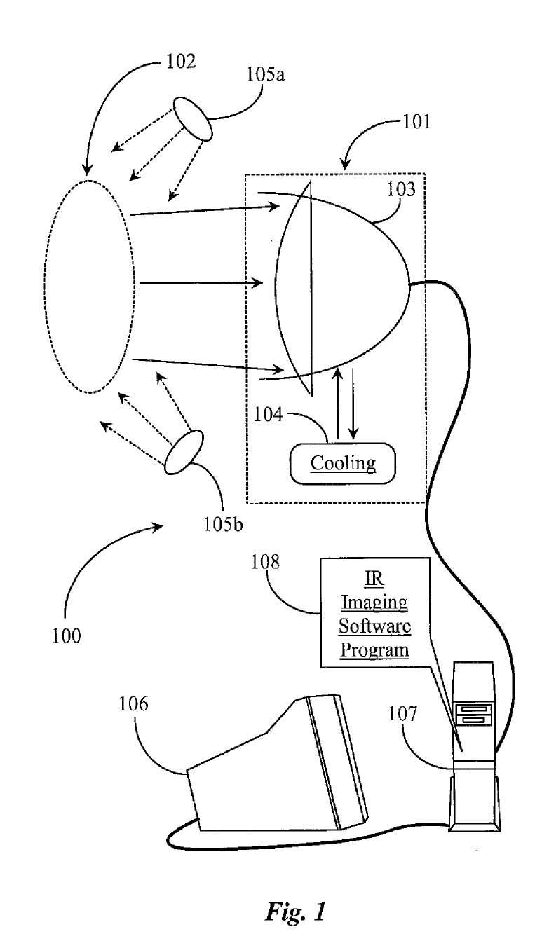

FIG. 1 is an architectural view of a digital thermal imaging system 100 according to an embodiment of the present invention. System 100 is adapted as a thermal medical imaging system that may be used to detect and monitor visible or non-visible wounds. The type of imaging performed may be classed as no-contact thermal imaging using an infrared heat detector unit 101, which may be an infrared camera or a digital camera also equipped with infrared sensors and a mode for thermal imaging. Thermal detection unit 101 may be referred to in this specification as an infrared camera 103 having a cooling cell 104.

In one embodiment of the present invention, camera 103 is cooled using some form of cooling mechanism 104. Mechanism or cell 104 may be a thermoelectric cooling cell, a forced air unit, or a cryogenic or water-filled unit. As described further above in the background section, cameras that are cooled tend to be the better resolution cameras. Cooling is not, however required in order to practice the present invention. It is optional.

Camera 103 may be mounted on a track or an adjustable slide-bar such as on a goniometric track (not illustrated here) for measuring angles. In one embodiment camera 103 may be a hand-held camera. Camera 103 is typically placed close to but not contacting a targeted tissue area illustrated here by a broken boundary as tissue area 102, which may encompass a visible or non-visible wound for example. In this example there is a single camera or infrared detector in unit or system 104. However, this should not be construed a limitation as there may be more than one or several detectors or cameras like camera 103 included in a thermal imaging system such as system 104 with each detector or camera capable of producing an independent image stream. An embodiment such as this is described in more detail later in this specification.

In a preferred embodiment the system of the present invention is sensitive to near infrared, medium infrared and far infrared spectrums. Near infrared is from 0.07 microns to 2.0 microns while medium is 2.0 microns to about 4.0 microns and far infrared is 4.0 microns and above. The human body can emit radiation in the far range to 9.0 microns. In the most preferred embodiment, the system of the invention is sensitive to emissions as high as 9.0 microns.

Infrared detection unit 101 or more specifically camera 103 has a cable input connection to a computer tower processor 107, which in turn is cabled to a monitor 106 capable of displaying thermal images from camera 103. In this example, camera 103 functions as an input peripheral device connected to the computer system via a cable such as a universal serial bus cable (USB) or some other data transmission cable such as a fire wire cable (Apple Computer), a Super Video (S-Video) cable or Institute of Electrical and Electronic Engineers (IEEE) cable for example.

An infrared imaging software program 108 is provided on a removable or static digital medium generic to or provided and made accessible to computer tower 107 and is executable there from by a user exercising input command capability via a keyboard or other computer input mechanism (not illustrated). Program 108 may contain a graphical user interface that may enable a user operating imaging system or unit 101 to make certain configurations that enable or disable certain aspects of thermal imaging performed by the system. In one embodiment a GUI of program 108 allows different resolution settings to be selected for different types of wounds to be imaged. Other parameters such as lighting or no lighting, color saturation levels, image size, and so on can be regulated.

In one embodiment the interface also enables certain adjustments to image or signal processing functions. For example in one aspect of monitoring a wound, periodic imaging of the wound may be ordered over a recovery period. As new snapshots of thermal imaging are taken they may be automatically inserted into a progression of shots taken since the wound was detected. By analyzing the separate shots over the timeline of the wound, the rate of healing of the wound may be determined verses time. The healing rate may be determined adjunct to treatment regimens used so as to observe an acceleration of the healing rate or a deceleration of the healing rate over time. This is just one example of possible configuration options a GUI of SW 108 may provide to a user of the system. Digital signal processing may be performed onboard system 101 to a certain extent and onboard processing tower 107 as required to provide analytic results consistent with one time thermal imaging and prognosis data relative to multiple thermal imaging sessions of a same tissue area over time.

It is noted herein that thermal imaging using system 101 may include the use of one or more specified light sources such as light sources 105a and 105b illustrated in this example. Light sources 105a or 105b may be polychromatic or monochromatic light sources cable of executing filtered light. Use of light sources 105a and 105b during a thermal imaging session may be for the purpose of cancelling out ambient light and, or to maximize imaging contrast. Illuminating the wound site during imaging may also allow techniques such as fluoroscopy where a dye is injected or applied topically and fluorescence is measured when irradiated. Bandwidth filters may be provided with light sources selection options so that specific bandwidths may be selected. Light sources 105a and 105b may be part of apparatus generic to a patient staging area for thermal imaging or they may be installable on to unit 101. In one embodiment where the light sources are installed on to system 101, color selections and bandwidth filters may be made through the GUI of program 108 and effected remotely over the connection cable.

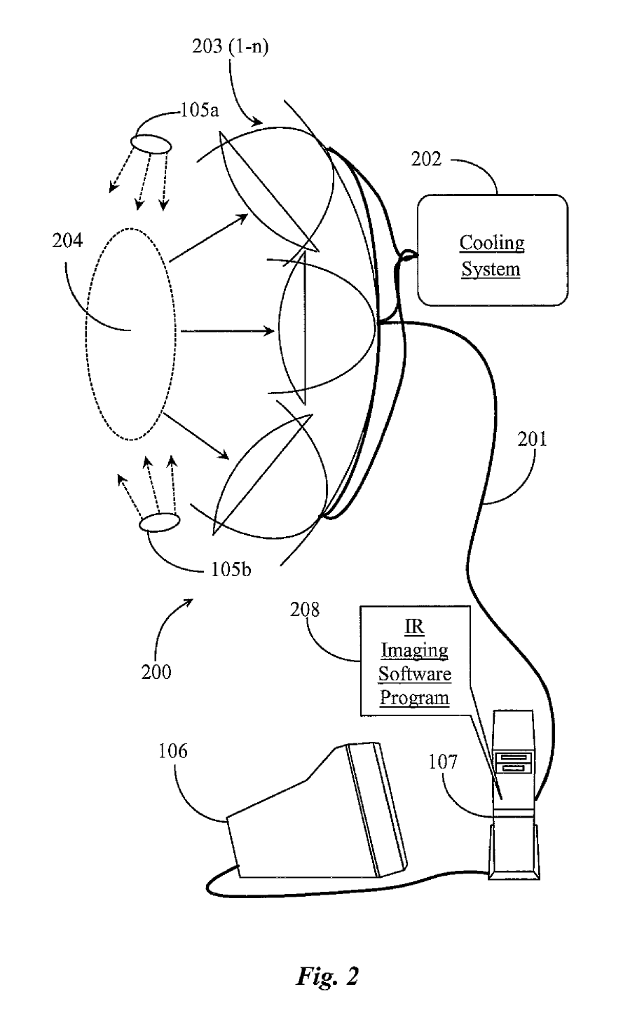

FIG. 2 is an architectural view of a digital thermal imaging system 200 according to another embodiment of the present invention. Architecture 200 is very similar to architecture 100 described above with the exception that there is more than one thermal imaging detector or camera provided within the thermal imaging system.

In system 200 a thermal imaging system comprises multiple infrared imaging cameras or detectors 203 (1-n). Cameras 203 1-n are mounted to a goniometric track and each camera position is adjustable along the track so as to measure or detect electromagnetic thermal radiation from a different and recordable angles. A targeted tissue area 204 emanates electromagnetic radiation picked up as thermal radiation by the infrared detectors. This is the same case as described in FIG. 1 except that there are multiple cameras or detectors receiving the electromagnetic radiation from different angles due to their current positions on the goniometric track. Light sources 105a and 105b may also be present and used to cancel out ambient light and/or to improve image contrasting.

Cameras 203 (1-n) share a single cooling system 202. A cable 201 is provided to tether the system to the computing system comprising computer tower 107 and monitor 106. A software program 208 is provided on digital media generic to or accessible to tower 107 and may be analogous to the SW 108 describe above except for an enhancement for incorporation into the processing of multiple cameras all producing an image stream of the same location from different angles.

In this particular example, each of cameras 203 (1-n) has a different “view” of the wound and more particularly, the thermal radiation pattern surrounding the wound. Each camera therefore has a separate signal of image data unique to its position of detection. All of the separate signals may be combined into one signal expressing the values of all of the separate cameras. In this way a more complete picture of the thermal map of the wound emerges, one that is tri-dimensional instead of one or two dimensional.

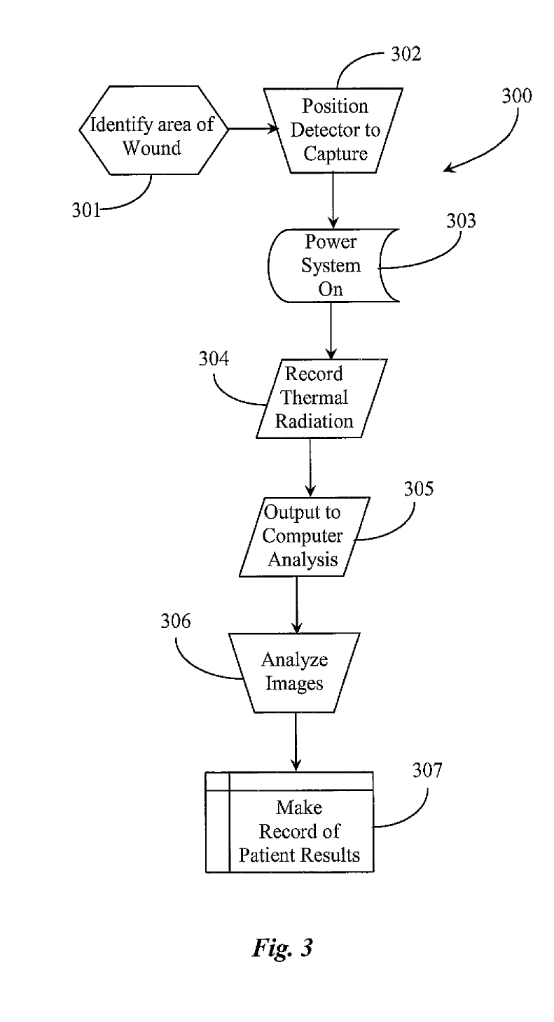

FIG. 3 is a process flow diagram illustrating steps 300 for thermal imaging of a wound according to one embodiment of the present invention. Steps 300 represent a simple process for creating and recording thermo-graphic images of a target tissue area. At step 301 the specific area of the wound is identified by the system. In this step the wound may be a visible wound or the wound may not be readily visible to the human eye. The system may be adapted to find an invisible wound by scanning over the general area of the wound and detecting via thermal imaging, the exact location and size of the wound. An example of this might be looking for and detecting an internal ulcer or an infection site not visible on the outside of the body.

Of course, if the wound is plainly visible or pre-diagnosed, visual methods may be sufficient to locate the wound for the purpose of positioning the camera there over as described in the next step 302. At step 302 the camera or detector is positioned very close to the wound but not in intimate contact with the wound. The distance from the wound and the detection system may vary somewhat within a range of about a few centimeters to a few decimeters. For wounds that are internal and some thickness away from the epithelial layer beyond a prescribed distance, for example, the system may actually contact the skin of a patient and may be pressed closer to an internal wound or infection site.

At step 303, the system may be powered on if it is not already powered on. There may be a power switch on the system or it may be powered on from the computer system.

At step 304 the thermal radiation emanating from the wound site is recorded. In this step, electromagnetic radiation emanating from healthy tissue surrounding the wound site may also be recorded so that the system may have a baseline reading for comparison. Output from the thermal imaging unit is sent as a digital signal in step 305 to computer analysis software residing on a computing system in removable or static digital media. At step 306 the images captured during thermal imaging are analyzed and diagnostic results are rendered through algorithm and routines designed for the purpose. At step 307 a record may be made of patient results. Ongoing treatment of a wound may be monitored and repetitious thermal imaging sessions may be conducted, the results thereof calculated over time thereby providing information relative to success of specific treatments or regimens.

One with skill in the art will appreciate that this processes is basic but may include more steps without departing from the spirit and scope of the invention. For example, a process step for canceling out ambient light or improving imaging contrast using one or more light sources may be included into the exemplary process. Other procedural steps may be inserted into this basic process depending on the type of wound the system is imaging. The process for thermal imaging of a diabetic ulcer may include more steps than one for imaging an external flesh wound such as a burn wound for example.

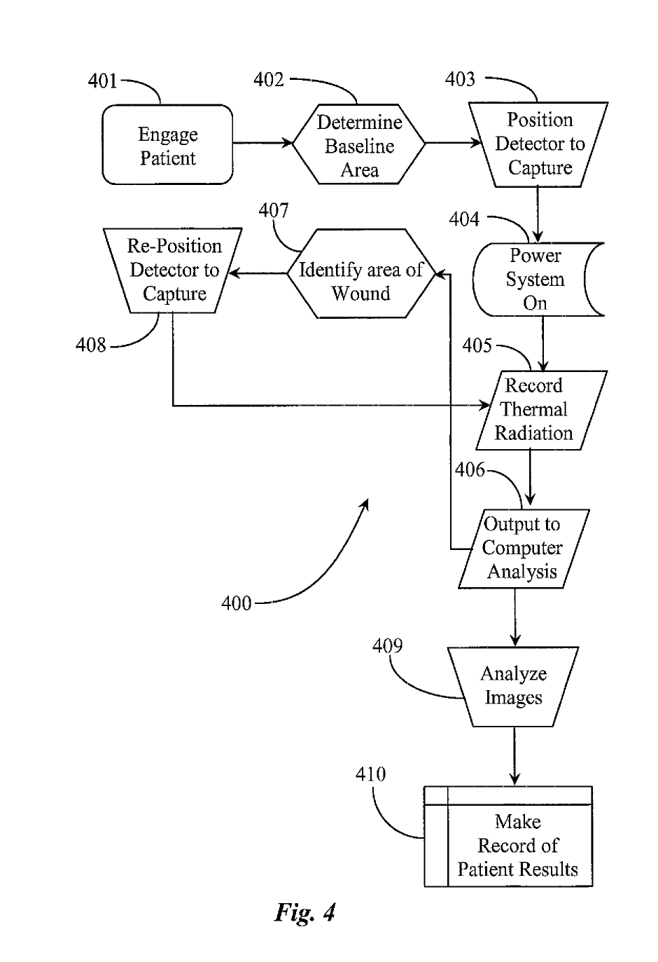

FIG. 4 is a process flow chart illustrating steps 400 for thermal imaging according to another embodiment of the present invention. At step 401, a patient with a wound for thermal imaging diagnosis is engaged. At step 402, a baseline thermal Image or images are taken of healthy tissue to determine a healthy thermal pattern with which to compare thermal images taken from the wound area. In one embodiment, a hand-held thermal imaging system is used to take the images for the baseline reading. In another embodiment, the imaging system may be fixed on a stand or other stable structure and the patient may position themselves for the imaging session.

At step 403 the imaging camera or detector (there may be more than one) is positioned or adjusted to detect the electromagnetic radiating from the healthy tissue. At step 404, a practitioner or other authorized personnel such as a nurse or doctor or imaging specialist may power the thermal imaging system to on. This step may be performed at the location of the camera/detector or from the main computer tower. At step 405 the system records the thermal imagery which may be one or more than one snapshot of the radiating pattern expressed in the form of a color thermography image. A temperature scale may also be available as part of a thermography image to indicate what colors correspond with what temperature ranges.

Image data from the infrared camera or detector is output at step 406 to a computer analysis program installed on a host computing appliance analogous to the computer and monitor system described further above. At this step data from healthy tissue is recorded and available to the system for comparison. The process may then move on to step 407 where the area of the wound is identified for imaging purposes. The wound area may be visible to a practitioner or it may be invisible (underneath the skin). Step 407 may involve using one or more infrared cameras to “look” for a spike in radiation emanating from an invisible so that correct thermal thermograph positioning may be undertaken.

In any case at step 408 the detector or camera is positioned or repositioned, in the case of previous baseline reading, so that thermography images can be rendered of the targeted ground area. The process then resolves back to step 405 where the system records the thermal radiation as one or more than one thermal image. The following steps are the same as those or thermal imaging of healthy tissue. For example, at 406 the data is output to a computer analysis program analogous to SW 108 or SW 208 previously described. At step 409 the images taken of both the healthy tissue and the wound tissue are analyzed. The process may involve comparing the baseline image from the healthy tissue to the image from the wound tissue to determine the amount of difference in radiation between the two. If the baseline reading is stable and fairly constant it can be used as a marker of what the wound radiation needs to be lowered to. Typically speaking a wound may emanate a warmer radiation pattern than healthy tissue because of several factors. One is that increased blood flow created during the healing process may raise temperatures slightly around the wound site.

Cell growth such as within a wound will cause more energy to be emitted. The emitted energy is what is important to measure to deduce the state of a wound against a baseline reading of healthy energy. On the other hand for some wounds Infrared signature can be caused by colder wounds such as chronic ulcers. For some wounds blood flow is actually reduced or does not reach the wound effectively and therefore the local radiation energy is below what a healthy signature might be.

The emitted radiation must be deduced through algorithm during image analysis as both reflective radiation and transmitted radiation may also be present and detected by the imaging device or camera. Algorithms for image comparison and those for noise cancellation may be provided to obtain accurate thermal readings. In wound monitoring where periodic sessions are conducted the important data refers to the thermal response of the wound tissue to various treatments as a progression over time. In this way various treatments may be compared to one another for effectiveness. For example, for an infected wound, different antibiotics may produce different levels of metabolic activity in the wound tissue thus a different level of emitted radiation from the wound site.

At step 410 the prognosis data is made a record for the patient. The patient data may include the actual thermal images in the form of j-peg or other image compression formats. It will be apparent to one with skill in the art of wound treatment and recovery that there are a variety of things that could increase electromagnetic thermal energy from a wound site. Elevations or spikes in thermal energy emitted from a wound site may be caused by increased blood flow, bacterial division, cell growth, antibody activity or other metabolic changes occurring over relatively short periods of time during the progression of the wound. Therefore, a spike in thermal energy emitted from a wound site can mean completely different things depending on the type of wound and treatment of the wound. SW routines used in analyzing thermal imaging data may vary according to wound type and expected treatments.

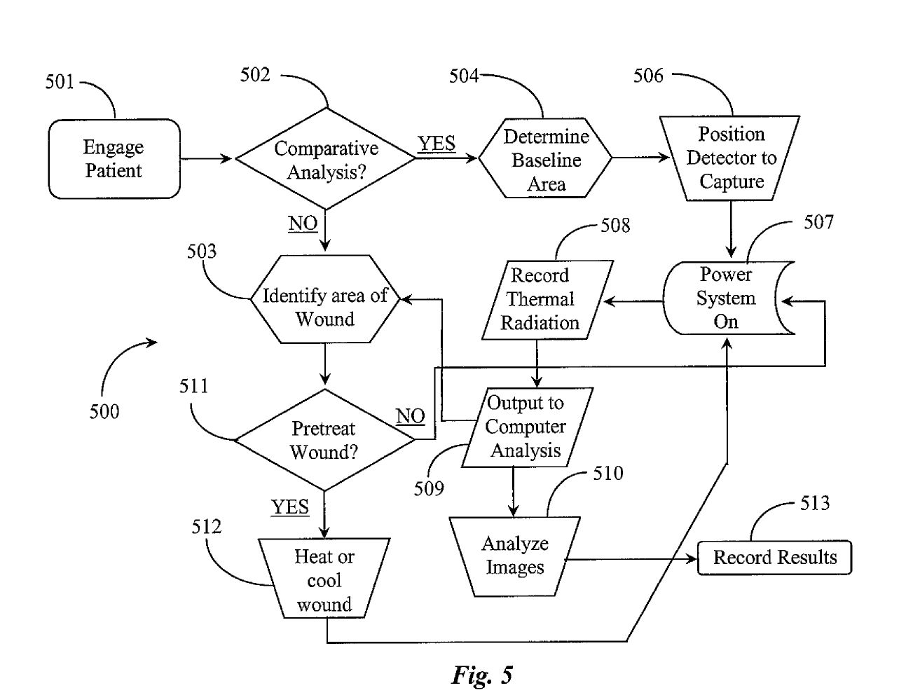

FIG. 5 is a process flow chart illustrating steps 500 for thermal imaging according to another embodiment of the invention. At step 501 the patient having a wound to treat is engaged. It may be determined by a practitioner or other authorized medical worker whether a comparative analysis of wound thermography against a baseline thermography will be conducted as part of the thermal imaging process at step 502. If at step 502 it is determined that a comparative analysis will be performed then at step 504 the practitioner may determine an area of healthy tissue from which to take baseline readings from to use for comparison against wound thermography.

The process may then move to step 506 where the camera or detector is positioned to capture thermal radiation emanating from the healthy tissue selected for baseline reading. The system may be powered on at step 507. At step 508, the thermal imaging is performed. At this step one or more thermal images may be recorded. At step 509 the data is output from the imaging device to the computing system for use in computer based analysis.

The process may move on to step 503 where the area of the wound on the patient is identified for thermal imaging of the wound. The process may also move directly to step 503 if at step 502 it is decided that a comparative analysis will not be performed. After the wound location has been identified for imaging purposes it may be determined at step 511 whether the wound will be pre-treated before imaging commences. Pre-treatment of a wound may involve purposeful heating of or cooling of the wound area before imaging. This may be for the purpose of beginning thermal recording at a preconditioned level of thermal emission from the wound. If at step 511 it is determined that the wound will be pre-treated before thermal imaging then at step 512 the wound site may be heated or cooled accordingly.

If the wound site is on an extremity like a foot or hand then a special temperature control heating or cooling boot or glove may be worn for a prescribed period of time to obtain the desired pre-condition. Heat or cold displacement or decay rate from a wound site will be fairly consistent after the heating or cooling stimulus is removed. Deviations from the typical displacement rate in either direction can point to other sourced electromagnetic radiation such as emitted radiation that can be isolated and measured.

In one embodiment the temperature controlled glove or boot is used to produce an artificial temperature state at the wound site the transition from which back to the actual thermal state of the wound is monitored with respect to time of completion of the transition and analyzed for comparative results. The changes in thermal transition time and rate of thermal decline or increase can be mapped comparatively to other sessions conducted at other times to isolate and identify prognostic data.

If at step 511 it is determined that the wound will not be pretreated then the process may resolve to step 507 where the infrared imaging system is powered on. In the case of wound pretreatment at step 512 through heating or cooling, the process may also resolve to step 507 where the system is then powered on. In one embodiment the system is already powered on or remains powered on indefinitely. At step 508 the thermal radiation imaging of the wound site commences whether the wound is pretreated or not. At step 509 the image data is output to computer analysis. At step 510 the image data is analyzed and may be compared to any other data already acquired such as baseline data. At step 513 the results may be recorded and made part of the patient record. Pretreatment of a wound may include some other type of treatment that deviates from conventional heating or cooling and that may also have an affect on the overall level of radiation detected by one or more cameras.

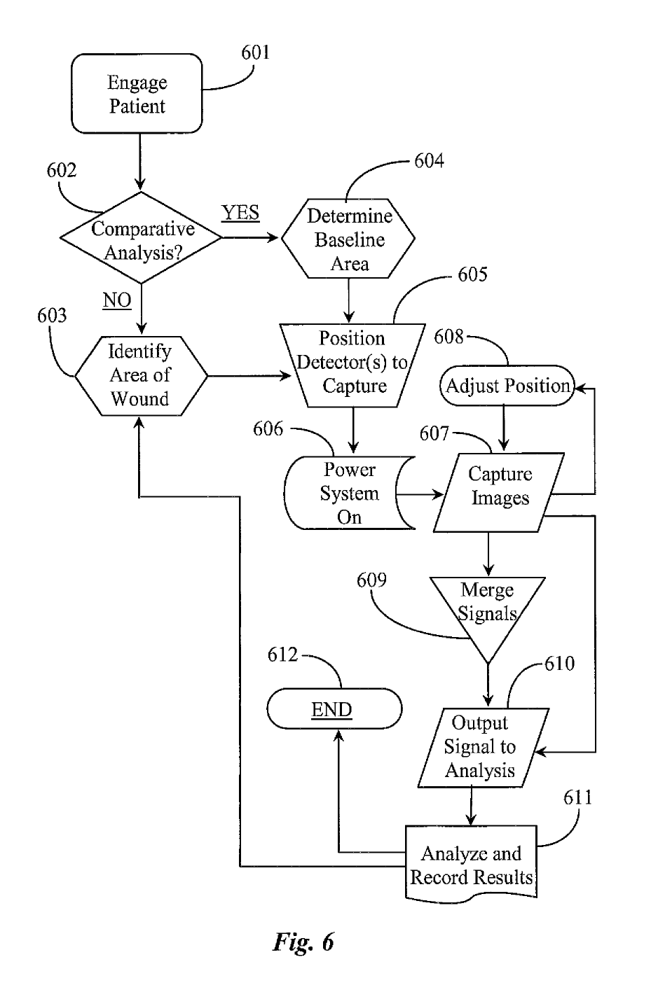

FIG. 6 is a process flow chart illustrating steps for thermal imaging according to yet another embodiment. At step 601 the patient is engaged by a practitioner who will perform or at least set up the imaging session. At step 602 it is determined if a comparative analysis will be done. If it is determined that a comparative analysis will be performed at step 602, then at step 604 a baseline area is determined from whence a radiation pattern for healthy tissue can be deduced. If no comparative analysis is to be performed then the process moves to step 603 to identify the area of the wound to be imaged.

In either case of step 602 at step 605 the cameras or detectors are positioned to capture thermal images. In this case there are more than two cameras or detectors and they can simultaneously render images from different viewpoints. These cameras may be mounted on a goniometric arc or path where the individual positions of the imaging devices can be changed at will.

In the case where a baseline reading will be taken at step 604, then steps 605 and 606 are executed as described earlier in other process flows. At step 607 the system captures the thermal images. In the case of imaging healthy tissue for a baseline reading, multiple cameras may or may not be used and position adjustment of cameras in step 608 may not be necessary. Likewise in the case of healthy tissue imaging, step 609 may be skipped and a single image signal may be output to analysis SW (that of the healthy tissue). The process may the resolve back to step 603 where the wound site is then identified for subsequent imaging of the wound. In the case of no comparative analysis then the process may move from step 601 to 603.

In the case of thermal imaging of the wound, at step 605 the cameras are positioned about a goniometer track or other apparatus for the purpose of imaging from different angles relative to the wound site. In one embodiment multiple devices are used to render streams that when combined might produce a tri-dimensional model of the wound radiation pattern. In this way depth of the wound and other information may be gathered that would not otherwise be available in a one dimensional thermal image.

At step 606 the system is powered on and at step 607 thermal images are captured from the different angles of mounted cameras. It is noted herein that one camera may record thermal images from one position and then be moved to a second position for a subsequent image capture. Therefore step 608 is added for adjusting the position of a camera after a previous image capture session in order to commence a next image capture session. In the case of several cameras, all of the cameras may be moved to a next position and further imaging may commence.

In the case of multiple thermal imaging devices step 609 is provided for merging or combining thermal image data signals output from the cameras before outputting a combined signal to analysis at step 610. At step 611 then all of the imaging data may be analyzed including comparison against baseline data to produce useable results which can be recorded. The process may end at step 612 after clean results are recorded. It is noted herein that over multiple image sessions, previously recorded data results may be used in any of the algorithms supplied with SW to help generate prognosis data over time such as rate of tissue regeneration for a particular wound. There are many possibilities.

It will be apparent to one with skill in the art that the thermal imaging system and methods of the invention may be provided using some or all of the mentioned features and components without departing from the spirit and scope of the present invention. It will also be apparent to the skilled artisan that the embodiments described above are exemplary of inventions that may have far greater scope than any of the singular descriptions. There may be many alterations made in the descriptions without departing from the spirit and scope of the present invention.

What is claimed is:

1. A system for detecting and/or monitoring damaged tissue comprising:
a thermal imaging device;
a data processing device including a digital media generic to or associated there with connected to the thermal imaging device;
an image display device connected to or integrated with the data processing device; and
a graphics user interface resident on the digital media of the data processing device and executable to display on the image display device, the interface enabling user configuration of various aspects of thermal imaging and data analysis functions.

2. The system of claim 1 wherein the imaging device is a digital camera with at least one infrared detector, the data processing device is a computer processor tower and the display device is a connected computer monitor.

3. The system of claim 1 wherein the damaged tissue is externally visible.

4. The system of claim 1 wherein the damaged tissue is underneath the skin and not externally visible.

5. The system of claim 4 used to detect the damaged tissue.

6. The system of claim 1 wherein the thermal imaging device is sensitive to thermal radiation from 0.07 microns in the near infrared range up to 9 microns in the far infrared range.

7. The system of claim 1 wherein the connection between the processing device and the thermal imaging device is a data cable.

8. The system of claim 7 wherein the cable is one of a universal serial bus cable, a fire wire cable, an Institute of Electrical and Electronic Engineers (IEEE) cable or a Super Video (S-Video) cable.

9. The system of claim 1 further including:
at least one additional imaging device; and
a mounting bracket or mechanism for facilitating adjustable mounting of the imaging devices.

10. The system of claim 9 wherein the mechanism to which the cameras are mounted to is a goniometer track or device.

11. The system of claim 9 wherein the imaging devices are digital cameras with at least one infrared detector, the data processing device is a computer processor tower and the display device is a connected computer monitor.

12. The system of claim 9 wherein the damaged tissue is externally visible.

13. The system of claim 9 wherein the damaged tissue is underneath the skin and not visible.

14. The system of claim 9 used to detect the damaged tissue.

15. The system of claim 9 wherein the thermal imaging device is sensitive to thermal radiation from 0.07 microns in the near infrared range up to 9 microns in the far infrared range.

16. A method for thermal imaging of damaged tissue comprising steps:
(a) powering on a thermal imaging system, the system including at least one thermal imaging device;
(b) locating the damaged tissue to be imaged;
(c) positioning the imaging device or devices over the tissue to be imaged; and
(d) recording the thermal images.

17. The method of claim 16 wherein in step (a) the thermal imaging system also includes a computer processing tower, a connected monitor, and a graphics user interface.

18. The method of claim 16 wherein the system is used to locate the damaged tissue, the tissue not visible to the operator of the system.

19. The method of claim 16 further including a step between step (b) and step (c) for pre-treating the wound using a temperature controlled glove or boot.

20. The method of claim 16 wherein in step (c) the devices are positioned around the wound on a goniometer track and at step (d) multiple image recordings are made from the devices at different positions.

21. The system of claim 1 wherein the connection between the thermal imaging device and the data processing device is a wireless connection the imaging data transmitted to the data processing system from the thermal imaging device over the connection.

22. The method of claim 19 wherein the glove or boot is also used to enhance a thermal signature by reducing noise.

23. The method of claim 19 wherein the temperature controlled glove or boot is used to produce an artificial temperature state at the wound site the transition from which back to the actual thermal state of the wound is monitored with respect to time of completion of the transition and analyzed for comparative results.

24. The system of claim 1 wherein the damaged tissue is illuminated during thermal imaging to improve image contrast.

25. The system of claim 9 wherein the damaged tissue is illuminated during thermal imaging to improve image contrast.

26. The method of claim 16 wherein a step is added between steps (c) and (d) for illuminating the damaged tissue to improve image contrast.

27. The system of claim 1 wherein a temperature controlled glove or boot is used to produce an artificial temperature state at the wound site the transition from which back to the actual thermal state of the wound is monitored with respect to time of completion of the transition and analyzed for comparative results.

29. The system of claim 9 wherein a temperature controlled glove or boot is used to produce an artificial temperature state at the wound site the transition from which back to the actual thermal state of the wound is monitored with respect to time of completion of the transition and analyzed for comparative results.
